(19) United States
(12) Patent Application Publication
(10) Pub. No.: US 2017/0164869 A1
(43) Pub. Date: Jun. 15, 2017

(54) SYSTEMS AND METHODS FOR TRACKING AND DISPLAYING ENDOSCOPE SHAPE AND DISTAL END ORIENTATION

(71) Applicant: National University of Singapore, Singapore (SG)

(72) Inventors: Tswen Wen Victor Lee, Singapore (SG); Wee Chuan Melvin Loh, Singapore (SG); Tsui Ying Rachel Hong, Singapore (SG); Siang Lin Yeow, Singapore (SG); Jing Ze Li, Singapore (SG)

(21) Appl. No.: 15/117,000

(22) PCT Filed: Feb. 5, 2015
(86) PCT No.: PCT/SG2015/000030

§ 371 Date: Aug. 5, 2016

Related U.S. Application Data
(60) Provisional application No. 61/936,037, filed on Feb. 5, 2014.

(51) Int. Cl. A61B 5/06; A61B 1/00; A61B 5/00; A61B 1/005

(52) U.S. Cl. CPC A61B 5/062; A61B 1/005; A61B 5/067; A61B 1/00045; A61B 1/0011; A61B 5/742

(57) ABSTRACT

Systems and methods for tracking shape and orientation of an endoscope employ motion tracking sensors to track locations on the endoscope for use in determining real time shape and distal end orientation for display during navigation of the endoscope. An example system includes sensor units distributed along the endoscope and a control unit. The sensor units track motion of the endoscope locations and transmit resulting tracking data to a control unit. The control unit processes the tracking data to determine shape of the endoscope and orientation of the distal end of the endoscope. The control unit generates output to a display unit that causes the display unit to display one or more representations indicative of the shape of the endoscope and orientation of the distal end of the endoscope for reference by an endoscope operator during an endoscopic procedure.

### CROSS-REFERENCES TO RELATED APPLICATIONS

This application claims priority of and the benefit of U.S. Provisional Application No. 61/936,037, entitled “THREE DIMENSIONAL COMPASS ASSISTED NAVIGATION TO AUGMENT ENDO-LAPAROSCOPY,” filed Feb. 5, 2014, the full disclosure of which is incorporated herein by reference for all purposes.

### BACKGROUND

Endoscopy is used in a wide variety of patient examination procedures. For example, endoscopes are used to view the gastrointestinal tract (GI tract), the respiratory tract, the bile duct, the ear, the urinary track, the female reproductive system, as well as normally closed body cavities.

In certain applications, it can be difficult to properly maneuver an endoscope during insertion. For example, colonoscopy is one of the most frequently performed outpatient examination. Colonoscopy, however, is also one of the most technically demanding due to the potential for unpredictable looping of the endoscope during insertion due to the anatomy of the colon, which has characteristics that present challenges to the safe and successful advancement of the endoscope. For example, the colon is crumpled, convoluted, and stretchable with a very tortuous pathway, which includes several acute angles. These characteristics of the colon often leads to looping of the endoscope during advancement. Additionally, most of the length of the colon is mobile thereby providing no fixed points to provide counter traction during advancement. Furthermore, there are no obvious landmarks within the lumen of the colon, making it difficult for the surgeon to gauge the actual position and orientation of the endoscope. In summary, colonoscopy can be very unpredictable and counterintuitive to perform. As a result, full colonoscopic examination involving caecal intubation (the final landmark) occurs in approximately 85% of the time in most endoscopic units, which is not ideal.

During advancement and manipulation of the colonoscope in this difficult anatomy, the surgeon may cause the colonoscope to pitch about a lateral axis or roll about a longitudinal axis. Such rolling results in difficulty in relating manipulation input at the proximal end (where the surgeon is steering) to resulting movement of the distal end of the endoscope, as an image generated by the endoscope does not correspond to the orientation of the endoscope operator. As a result, the endoscope operator may attempt to conform the orientation of the endoscope to the operator's orientation by twisting the endoscope from the proximal end, in the clockwise or counter-clockwise direction. Such twisting, however, can result in increased looping of the endoscope if done in the wrong direction. Additionally, studies have shown that up to 70% of the time, loops are incorrectly diagnosed by the colonoscopist (see, e.g., Shah et al, “Magnetic imaging of colonoscopy: an audit of looping, accuracy & ancillary measures”, Gastroinestinal Endoscopy, 2000, v. 52, p. 1-8).

Controlling and steering the colonoscope is even more challenging to trainees and surgeons with less experience. Many of these inexperienced operators lack sufficient tactile discrimination to accurately gauge the orientation of the colonoscope and thus often rely on trial and error to advance the colonoscope. Studies have confirmed a direct correlation between increasing volume of an endoscopist's procedures with successful intubation rates. For example, among junior endoscopists, one study indicates that an annual volume of 200 procedures is required to maintain adequate competence (Harewood, “Relationship of colonoscopy completion rates and endoscopist features”, Digestive diseases & science, 2005, v. 50, p. 47-51). Lack of experience leads to increased procedural time and patient discomfort. The average procedural time for colonoscopy is about 20 minutes (see, e.g., Allen, “Patients' time investment in colonoscopy procedures”, AORN Journal, 2008). In the hands of an inexperienced endoscopist, colonoscopy may last from 30 minutes to an hour. Extended procedural time is also not the only cause of patient discomfort. Excessive stretching and looping of the colon may cause patients to experience abdominal pain and cramps, lightheadedness, nausea, and/or vomiting.

Thus, in view of the issues described above, there is a need to help surgeons advance endoscopes with higher success rates and shorter times.

### BRIEF SUMMARY

Systems and methods are provided for tracking and displaying real time endoscope shape and distal end orientation to an operator as an aid to the operator in advancing and maneuvering an endoscope during an endoscopic procedure. In many embodiments, the systems and methods utilize sensors that can be coupled with an existing endoscope that transmit position and orientation data to a processing unit, which determines real time shape and distal end orientation of the endoscope that is output for display to the endoscope operator. In many embodiments, the systems and methods can be used with existing endoscopes by coupling the sensors with the endoscope and utilizing a dedicated processing unit and a dedicated display.

Thus, in one aspect, an endoscope shape and distal end orientation tracking system is provided. The system includes a first sensor unit, a plurality of second sensor units, and a control unit. The first sensor unit is configured to be disposed at a distal end of an endoscope and generate position and orientation tracking data for the distal end of the endoscope. Each of the second sensor units is configured to be disposed a one of a corresponding plurality of locations along a length of the endoscope proximal to the distal end of the endoscope and generate position tracking data for the respective location. The control unit is configured to: (1) receive (a) the position and orientation tracking data generated by the first sensor unit for the distal end of the endoscope, and (b) the position tracking data for each of the respective locations by the respective second sensor units; (2) determine shape of the endoscope and orientation of the distal end of the endoscope based on the data generated by the first and second sensor units; and (3) generate output to a display unit that causes the display unit to display a representation of the shape of the endoscope and orientation of the distal end of the endoscope.

The first sensor unit and the second sensor units can include any suitable position and/or orientation tracking sensors to generate position and/or orientation tracking data. For example, The first sensor unit can include an accelerometer, a magnetometer, and a gyroscope that generate the position and orientation tracking data for the distal end of the endoscope. As another example, each of the plurality of second sensor units can include an accelerometer and a magnetometer that generate the position tracking data for the respective location.

The control unit can use any suitable algorithm for determining real time shape of the endoscope and orientation of the distal end of the endoscope. For example, the control unit can store calibration data used to determine the shape of the endoscope and orientation of the distal end of the endoscope from the data generated by the first sensor unit and the second sensor units. As another example, an initialization process can be used in which the endoscope, prior to insertion, is placed in a known shape and orientation and a correlation recorded between the know shape and orientation and corresponding data generated by the first sensor unit and the second sensor unit.

In many embodiments, the system includes one or more wireless transmitters to wirelessly transmit: (1) the position and orientation tracking data generated by the first sensor unit for the distal end of the endoscope, and (2) the position tracking data for the plurality of locations generated by the second sensor units. In such embodiments of the system, the control unit can include a wireless receiver to receive the data transmitted by the one or more wireless transmitters. In many embodiments of the system, each of the first sensor unit and the plurality of the second sensor units includes one of the wireless transmitters.

In many embodiments, the system includes an insertion wire assembly that includes an insertion wire, the first sensor unit coupled with the insertion wire, and the second sensor units coupled with the insertion wire. The insertion wire assembly can be configured for insertion into a working channel of the endoscope to position the first sensor unit adjacent to the distal end of the endoscope and each of the second sensor units at a respective one of the locations along the length of the endoscope. In many embodiments, the insertion wire assembly is removable from the working channel when the distal end of the endoscope is disposed within a patient (e.g., at the desired target location within the patient).

In many embodiments of the system, each of the first sensor unit and each of the second sensor units is a disposable units shaped for attachment to an external surface of the endoscope. Each of the first sensor unit and each of the second sensor units can include one of the wireless transmitters. Each of the first sensor unit and each of the second sensor units can include a battery.

The system can also be integrated into an endoscope when the endoscope is fabricated. For example, each of the first sensor unit and the plurality of the second sensor units can be embedded within an endoscope during manufacturing of the endoscope.

Any suitable display of the real time shape and orientation of the distal end of the endoscope can be employed. For example, the displayed representation of the shape of the endoscope and orientation of the distal end of the endoscope can indicate: (1) a longitudinal twist angle of the distal end of the endoscope relative to a reference twist angle, and (2) the amount of tilt of the distal end of the endoscope. In many embodiments of the system, the displayed representation of the orientation of the distal end of the endoscope displays the amount of tilt of the distal end of the endoscope via a representation that is rotated by an angle matching the longitudinal twist angle relative to a reference display angle. In many embodiments of the system, the displayed representation of the shape of the endoscope and orientation of the distal end of the endoscope includes a three-dimensional representation of the distal end of the endoscope as viewed from a viewpoint that varies to reflect changes in the orientation of the distal end of the endoscope.

In another aspect, a method is provided for tracking shape and distal end orientation of an endoscope. The method includes generating position and orientation tracking data for the distal end of an endoscope with a first sensor unit disposed at the distal end of the endoscope. The position and orientation tracking data for the distal end of the endoscope is transmitted from the first sensor unit to a control unit. Position tracking data for each of a plurality of locations along a length of the endoscope proximal to the distal end of the endoscope is generated with a plurality of second sensors. Each of the second sensors is disposed at a respective one of the locations along the length of the endoscope. The position tracking data for the locations along the length of the endoscope is transmitted from the second sensors to the control unit. The position and orientation tracking data for the distal end of the endoscope and the position tracking data for the locations along the length of the endoscope are processed with a control unit to determine shape of the endoscope and orientation of the distal end of the endoscope. Output to a display unit is generated that causes the display unit to display a representation of the shape of the endoscope and orientation of the distal end of the endoscope.

In many embodiments of the method, the first sensor unit and the second sensor units include suitable position and/or orientation tracking sensors to generate position and/or orientation tracking data. For example, generating position and orientation tracking data for the distal end of an endoscope can include: (1) measuring accelerations of the first sensor unit via an accelerometer included in the first sensor unit, and (2) measuring orientation of the first sensor unit via a magnetometer included in the first sensor unit and/or a gyroscope included in the first sensor unit. As another example, generating position tracking data for the locations along the length of the endoscope comprises measuring accelerations of each of the second sensor units via an accelerometer included in the respective second sensor unit.

In many embodiments of the method, the position and/or orientation data is wireless transmitted from the first sensor unit and/or the second sensor units to the control unit. For example, transmitting the position and orientation tracking data for the distal end of the endoscope from the first sensor unit to a control unit can include wireless transmitting the position and orientation tracking data from the first sensor unit and receiving the wirelessly transmitted position and orientation tracking data via a wireless receiver included in the control unit. As another example, transmitting the position tracking data for the locations along the length of the endoscope from the second sensors to the control unit can include wireless transmitting the position tracking data from the second sensor units and receiving the wirelessly transmitted position tracking data via a wireless receiver included in the control unit.

In many embodiments, the method includes inserting an insertion wire assembly into a working channel of the endoscope. The insertion wire assembly includes an insertion wire, the first sensor unit coupled with the insertion wire, and the second sensor units coupled with the insertion wire. In many embodiments of the method, the insertion wire assembly is configured for insertion into a working channel of the endoscope to position the first sensor unit adjacent to the distal end of the endoscope and each of the second sensor units at a respective one of the locations along the length of the endoscope. In many embodiments of the method, the insertion wire assembly is removable from the working channel when the distal end of the endoscope is disposed within a patient (e.g., at the desired target location within the patient).

In many embodiments, the method includes attaching the first sensor unit and the second sensor units to an exterior surface of the endoscope. In many embodiments, the method includes detaching the first sensor unit and the second sensor units from the exterior surface of the endoscope after using the endoscope to complete an endoscopic procedure.

In many embodiments of the method, a suitable display of the real time shape and orientation of the distal end of the endoscope is employed. For example, the method can include displaying a three-dimensional representation of the distal end of the endoscope as viewed from a viewpoint that varies to reflect changes in the orientation of the distal end of the endoscope.

### BRIEF DESCRIPTION OF THE DRAWINGS

FIG. 1 is a simplified schematic illustration of an endoscope shape and distal end orientation tracking system, in accordance with many embodiments.

FIG. 2 is a simplified schematic illustration of components of the system of FIG. 1, in accordance with many embodiments.

FIG. 3 illustrates an exemplary display of the shape of a deployed endoscope having sensor units disposed therewith and the orientation of the distal end of the endoscope, in accordance with many embodiments.

FIG. 4 illustrates a low-profile sensor unit attached to the exterior surface of an endoscope, in accordance with many embodiments.

FIG. 5 illustrates the shape and components of the low-profile sensor unit of FIG. 4, in accordance with many embodiments.

FIG. 6 illustrates an endoscope having low-profile sensor units attached thereto, in accordance with many embodiments.

FIG. 7 illustrates an insertion wire assembly configured for insertion into a working channel of an endoscope and including an insertion wire having sensor units attached thereto, in accordance with many embodiments.

FIG. 8 shows a graphical user interface display that includes a representation of the shape of a tracked endoscope, a representation indicative of the orientation of the tracked endoscope, and an image as seen by the distal end of the tracked endoscope, in accordance with many embodiments.

FIG. 9A through FIG. 9C shows a graphical user interface display that indicates amount of relative twist of the endoscope and the transverse angle of the distal end of the endoscope, in accordance with many embodiments.

FIG. 10A through FIG. 11C shows a graphical user interface display of a three-dimensional representation of the distal end of the endoscope as viewed from a viewpoint that varies to reflect changes in the orientation of the distal end of the endoscope, in accordance with many embodiments.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

In many embodiments of the systems and methods described herein, the shape of an endoscope and orientation of the distal end of the endoscope is tracked and displayed to aid to the operator of the endoscope. In many embodiments, the display provides a visual indication of how much the distal end of the endoscope has twisted and tilted during advancement. Such a display not only helps the endoscope operator with overcoming spatial disorientation, but also helps the endoscope operator with straightening of the endoscope correctly.

In many embodiments, the tracked shape and orientation of the distal end of the endoscope is used to display a representation to the endoscope operator that indicates the direction and angle of the distal end of the endoscope during an endoscopic procedure, for example, during colonoscopy. By displaying one or more representations of the shape of the endoscope and the orientation of the distal end of the endoscope relative to the endoscope operator, the ability of the operator to successfully navigate the endoscope during advancement is enhanced.

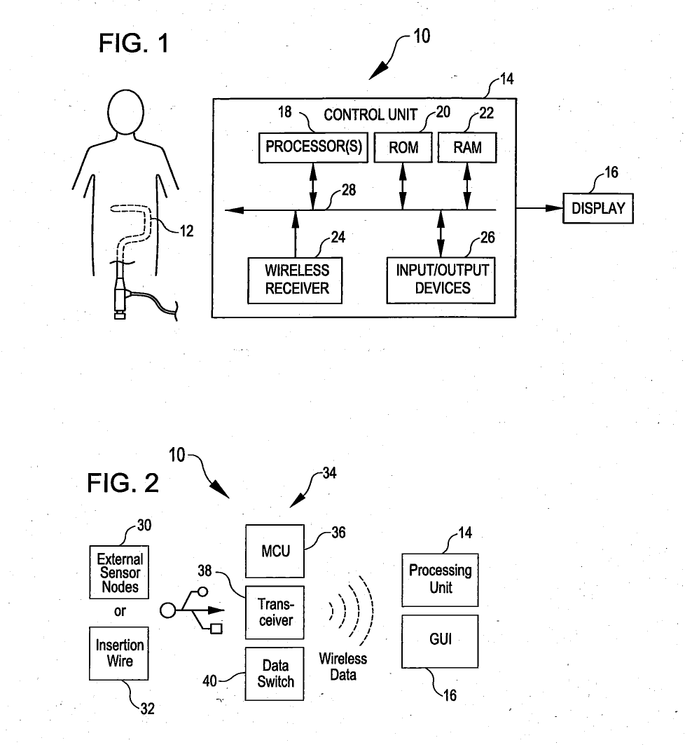

Turning now to the drawings, in which like reference numerals represent like parts throughout the several views, FIG. 1 shows a simplified schematic illustration of an endoscope shape and distal end orientation tracking system 10, in accordance with many embodiments. The system 10 includes an endoscope 12, a control unit 14, and a display 16. Motion sensing units are coupled with the endoscope 12 and used to generate position and orientation data used to track the shape of the endoscope 12 and the orientation of the distal end of the endoscope 12. The data generated by the motions sensing units coupled with the endoscope 12 is transmitted to the control unit 14, which processes the data to determine the real time shape of the endoscope 12 and the orientation of the distal end of the endoscope 12, which is then displayed via the display 16 as an aid to the endoscope operator in navigation of the endoscope 12 during an endoscopic procedure. Display 16 is not limited to a two-dimensional display monitor, and includes any suitable display device. For example, the display 16 can be configured to display the real time shape of the endoscope 12 and the orientation of the distal end of the endoscope using any suitable two-dimensional and/or three-dimensional display technology. Example two-dimensional and/or three-dimensional display technologies that can be employed to display the shape and distal end orientation of the endoscope 12 include, but are not limited to, three-dimensional image projection such as holographic image display and similar technologies, and displaying images on wearable devices such as a wearable glass display device, and other methods of displaying information indicative of the tracked shape and distal end orientation of the endoscope 12.

The control unit 14 can include any suitable combination of components to process the position and orientation data generated by the motion sensing units coupled with the endoscope 12 to determine the real time shape of the endoscope 12 and the orientation of the distal end of the endoscope 12 for display on the display 16. For example, in the illustrated embodiment, the control unit 14 includes one or more processors 18, read only memory (ROM) 20, random access memory (RAM) 22, a wireless receiver 24, one or more input devices 26, and a communication bus 28, which provides a communication interconnection path for the components of the controller 14. The ROM 20 can store basic operating system instructions for an operating system of the controller. The RAM 22 can store position and orientation data received from the motions sensing units coupled with the endoscope 12 and program instructions to process the position and orientation data to determine the real time shape of the endoscope 12 and the orientation of the distal end of the endoscope 12.

The RAM 22 can also store calibration data that correlates the position and orientation data with corresponding shape and orientation of the endoscope 12. For example, such correlation data can be generated by recording the position and orientation data generated by the motion sensing units during a calibration procedure in which the endoscope 12 is placed into one or more known shapes and orientation, thereby providing one or more known associations between the position and orientation data and specific known shapes and orientations of the endoscope 12. Such data can then be used to process subsequently received position and orientation data using known methods, including, for example, interpolation and/or extrapolation.

In many embodiments, the position and orientation data is wireless transmitted by the motion sensing units and received by the control unit via the wireless receiver 24. Any suitable transmission protocol can be used to transmit the position and orientation data to the wireless receiver 24. In alternate embodiments, the position and orientation data is non-wirelessly transmitted to the control unit 14 via one or more suitable wired communication paths.

FIG. 2 shows a simplified schematic illustration of components of the system 10, in accordance with many embodiments. As described herein, the system 10 includes the motion sensing units coupled with the endoscope 12, the control unit 14, and a graphical user interface (display 16). The motion sensing units can be implemented in any suitable manner, including but not limited to attachment to an exterior surface of an existing endoscope (diagrammatically illustrated in FIG. 2 as external sensor nodes 30). The motion sensing units can also be attached to an insertion wire 32, to which the motion sensing units are attached and which can be configured for removable insertion into a working channel of an endoscope so as to position the motion sensing units along the length of the endoscope as described herein. As yet another alternative, the motion sensing units can be integrated within an endoscope when the endoscope is manufactured.

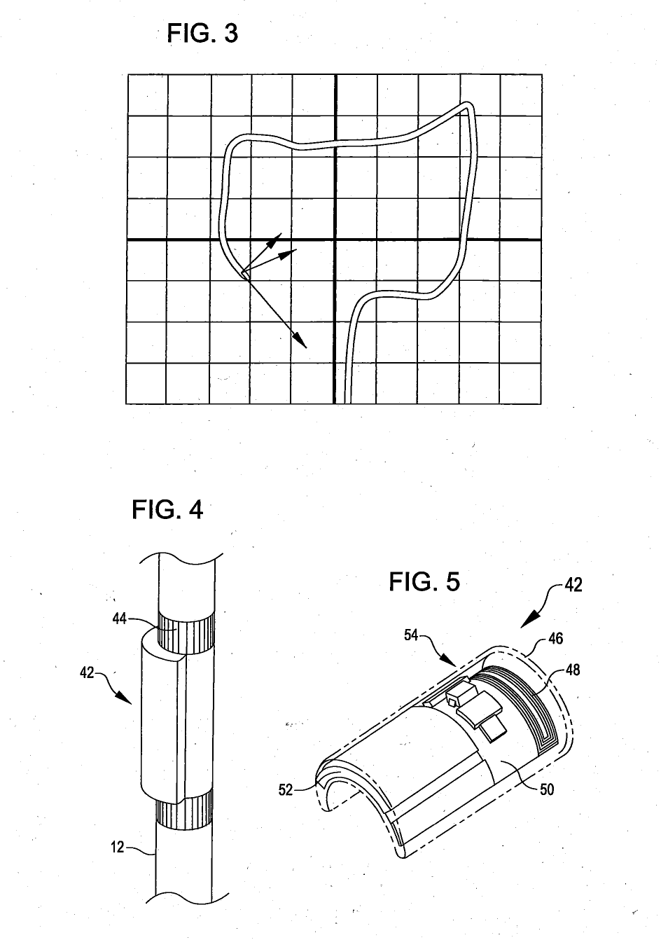

In the illustrated embodiment, the motion sensing units transmit data to a data transfer unit 34, which transmits the position and orientation data generated by the motion sensing units to the processing unit 14. In many embodiments, each of the motion sensing units includes a dedicated data transfer unit 34. In alternate embodiments, one or more data transfer units 34 is employed to transfer the data of one, more, or all of the motion sensing units to the control unit 14. In the illustrated embodiment, the data transfer unit 34 includes a micro controller unit 36, a transceiver 38, and a data switch 40. The data transfer unit 34 wirelessly transmits the position and orientation data generated by the motion sensing units to the control unit 14, which processes the position and orientation data to determine real time shape of the endoscope 12 and the orientation of the distal end of the endoscope 12 for display to the endoscope operator via the display 16. FIG. 3 illustrates an exemplary display of the shape of a deployed endoscope 12 having sensor units disposed therewith and the orientation of the distal end of the endoscope, in accordance with many embodiments.

FIG. 4 illustrates an embodiment of a low-profile motion sensing unit 42 attached to the exterior surface of an existing endoscope 12, in accordance with many embodiments. As illustrated, the low-profile motion sensing unit 42 has an curved profile shaped to mate with a curved external surface of the endoscope 12. In the illustrated embodiment, a thin flexible sheet 44 (e.g., a thin sheet of a suitable plastic) is tightly wrapped around the endoscope 12 and the motion sensing unit 42 is bonded to the sheet 44, thereby avoiding direct bonding between the motion sensing unit 42 and the endoscope 12 to enable easy removal of the motion sensing unit 42 from the endoscope 12 following completion of the endoscope procedure.

FIG. 5 illustrates the shape and components of the low-profile motion sensing unit 42, in accordance with many embodiments. In the illustrated embodiment, the motion sensing unit 42 includes a casing cover 46, an antenna 48, a flexible printed circuit board 50, a battery 52, and components 54 mounted on the circuit board 50. The components 54 can include an accelerometer, a magnetometer, a gyroscope, the micro controller unit 38, the transceiver 38, and the data switch 40. In many embodiments, the low-profile motion sensing unit 42 is configured to add between 2 to 3 mm in additional radial dimension to an existing endoscope 12.

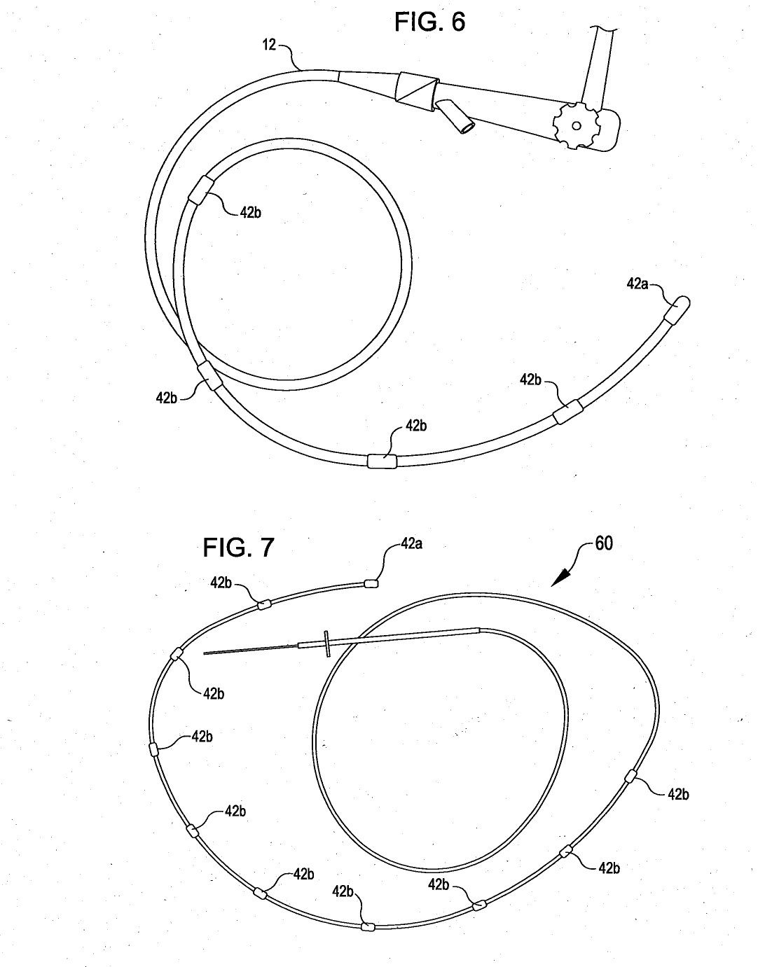

FIG. 6 illustrates an endoscope 12 having the low-profile sensor units 42 attached thereto, in accordance with many embodiments. The attached low-profile motion sensing units 42 include a first sensor unit 42a attached to the distal end of the endoscope 12 and a plurality of second sensor units 42b attached to and distributed along the length of the endoscope 12. In many embodiments, the first sensor unit 42a is configured to generate position and orientation tracking data that can be used to determine and track the position and orientation of the distal end of the endoscope 12. For example, the first sensor unit 42a can include an accelerometer, a magnetometer, and a gyroscope to generate the position and orientation tracking data for the distal end of the endoscope 12. In many embodiments, each of the second sensor units 42b is configured to generate position tracking data that can be used to determine and track the location along the endoscope 12 at which the respective second sensor 42b is attached. For example, each of the second sensor units 42b can include an accelerometer and a magnetometer to generate the position tracking data for the respective location along the endoscope 12. For each sensor unit 42a and 42b, motion sensor data is collected by external dedicated software. A sensor fusion algorithm has been developed to generate Quaternion representations from motion sensor data, including gyroscope, accelerometer and magnetometer readings. Conventional representations of orientation, including pitch, roll and yaw of each sensor units 42a and 42b are derived from the Quaternion representations in real-time. With known local spatial orientations of each sensor units 42b and prescribed distances between adjacent sensor units, interpolation of the directional vectors of each sensor units generates the shape of colonoscope 12 segmentations. Orientation and position information of the distal end of colonoscope 12, and the shape of the entire colonoscope 12 are hence computed in real-time, and visualization of the information is presented to user through display 16.

FIG. 7 illustrates an insertion wire assembly 60 configured for insertion into a working channel of an endoscope 12, in accordance with many embodiments. The insertion wire assembly 60 includes an insertion wire having the sensor units 42a, 42b attached thereto. Before the procedure, the insertion wire assembly 60 is inserted into the working channel at the proximal end of the endoscope 12. The display 16 can be affixed onto or near an existing endoscopy screen for the endoscope 12. In many embodiments, the sensor units 42a, 42b are configured to transmit the position and orientation data wirelessly to the control unit 14 for processing to display the shape of the endoscope 12 and the orientation of the distal end of the endoscope 12 on the display 16. As a result, in many embodiments, no additional steps may be needed to prepare the system. For example, when the system is used during a colonoscopy, the colonoscope operator can proceed according to normal protocol and insert the colonoscope into the rectum and advance the colonoscope through the large intestine.

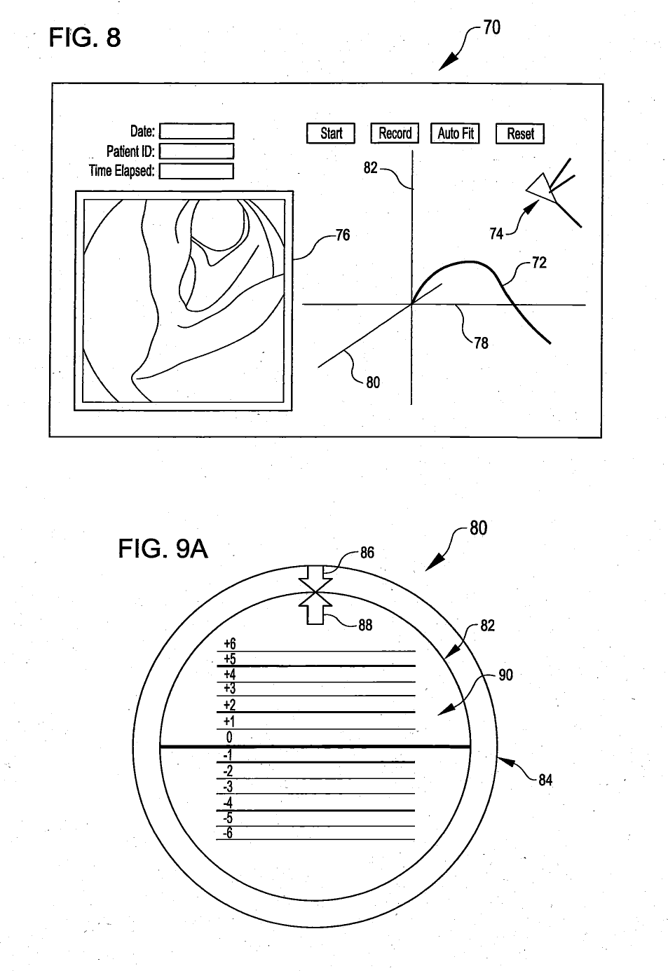

FIG. 8 shows a graphical user interface display 70 that includes a representation of the shape of a tracked endoscope 72, a representation indicative of the orientation of the tracked endoscope 74, and an image as seen by the distal end of the tracked endoscope 76, in accordance with many embodiments. The representation of the shape of the endoscope 72 and the representation indicative of the orientation of the tracked endoscope 74 are generated to be indicative of the real time shape of the endoscope 12 and the orientation of the distal end of the endoscope 12, respectively, as determined by the control unit 14. In the illustrated representations, the disposition of the length of the endoscope 12 relative to a reference axes 78, 80, 82 is displayed as the representation 72, and the orientation of the distal end of the endoscope 12 relative to the reference axes 78, 80, 82 is shown as the representation 74. During a colonoscopy procedure, the surgeon can use the graphical user interface display 70 to view the lining on the colon as well as steer the colonoscope.

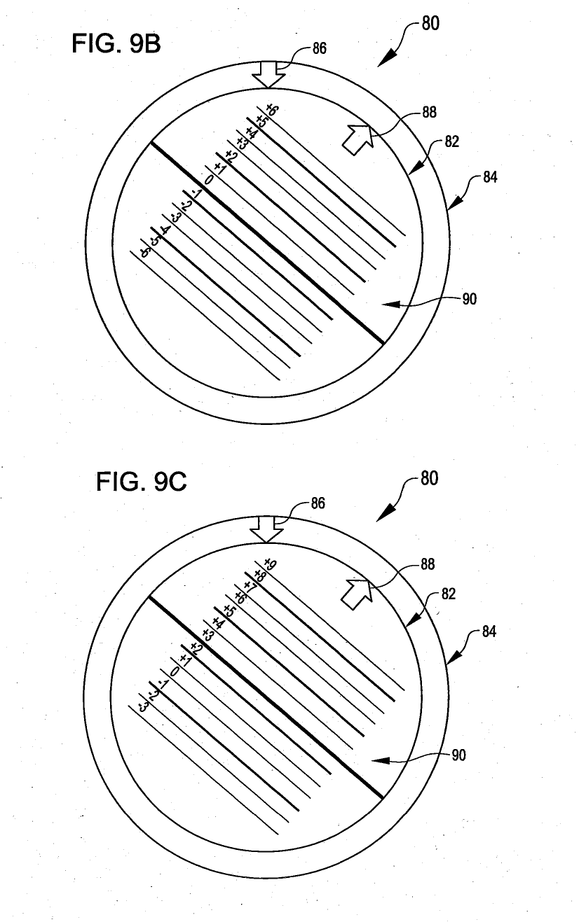

FIG. 9A through FIG. 9C shows a graphical user interface display 80, which is an alternative to the representation 74, that can be displayed on the display 16 to indicate amount of relative twist of the endoscope 12 and the transverse angle of the distal end of the endoscope 12, in accordance with many embodiments. The amount of relative twist of the endoscope 12 is shown via relative angular orientation difference between an inner display portion 82 relative to a fixed outer display portion 84, and between a fixed outer display reference arrow 86 that is part of the fixed outer display portion 84 and an inner display reference arrow 88 that rotates with the inner display portion 82. In FIG. 9A, the inner display arrow 88 is aligned with the fixed outer display reference arrow 86, thereby indicating that the endoscope 12 is not twisted relative to the reference endoscope twist orientation. In both FIG. 9B and FIG. 9C the inner display portion 82 is shown angled relative to the fixed outer display portion 84 as indicated by the misalignment of the inner display arrow 88 and the fixed outer display reference arrow 86, thereby indicating a relative twist of the endoscope 12 relative to the reference endoscope twist orientation. The relative twist of the endoscope 12 can be used by the endoscope operator to twist the endoscope 12 to be aligned with the reference endoscope twist orientation, thereby aligning the displayed image 76 with the reference endoscope twist orientation to reduce twist induced disorientation of the endoscope operator during navigation of the endoscope.

The inner display portion 82 of the graphical user interface display 80 includes a tilt indicator 90 that displays the angular tilt of the distal end of the endoscope 12. In both FIG. 9A and FIG. 9B, the tilt indicator 90 indicates zero tilt of the distal end of the endoscope 12. In FIG. 9C the tilt indicator 90 indicates a positive three degree tilt of the distal end of the endoscope 12. The indicated tilt of the distal end of the endoscope 12 can be used by the endoscope operator in combination with the displayed image 76 to adjust the tilt of the distal end of the endoscope 12 during navigation of the endoscope 12.

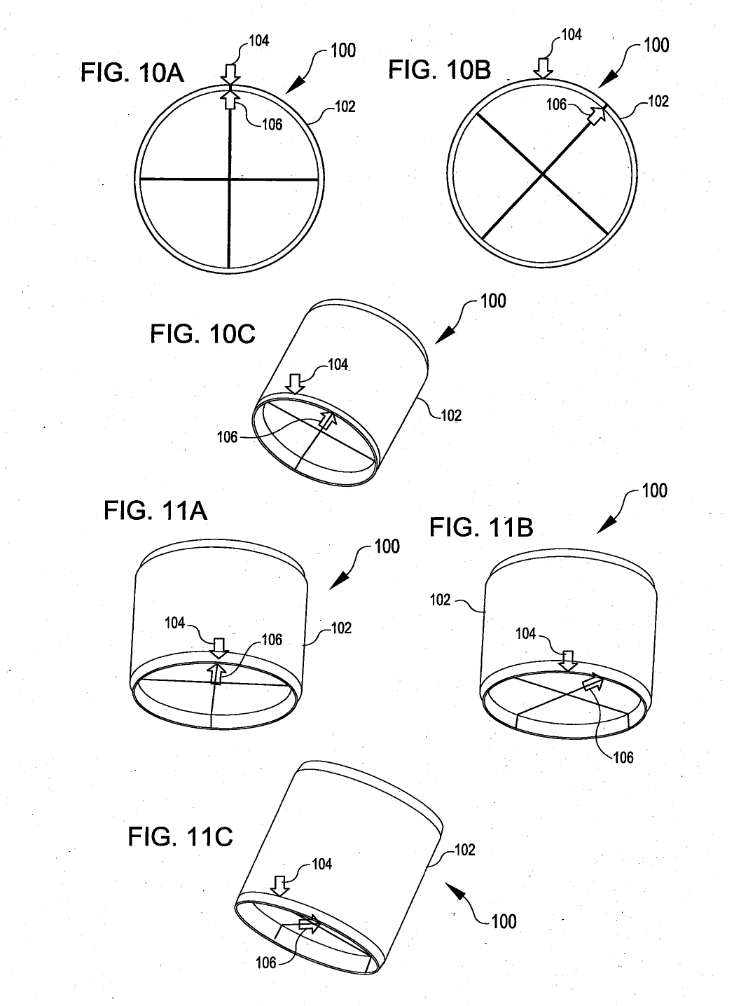

FIG. 10A through FIG. 11C shows a graphical user interface display 100, which is alternative to the representation 74. The display 100 includes of a three-dimensional representation 102 of the distal end of the endoscope 12 as viewed from a viewpoint that varies to reflect changes in the orientation of the distal end of the endoscope 12, in accordance with many embodiments. The graphical user interface display includes a fixed twist reference arrow 104 and a distal end twist reference arrow 106. Differences in relative alignment between the arrows 104, 106 is used to display amount of twist of the endoscope 12 relative to reference twist orientation. Additionally, the viewpoint from which the three dimensional representation 102 is shown is indicative of the three dimensional orientation of the distal end of the endoscope 12 relative to a reference orientation. For example, FIG. 10A shows the graphical user interface display 100 for zero relative twist of the distal end of the endoscope 12 and the orientation of the distal end of the endoscope 12 being aligned with the reference orientation. FIG. 10B shows the distal end aligned with the reference orientation and twisted clockwise relative to the reference twist orientation. FIG. 10C shows the distal end of the endoscope 12 twisted relative to the reference twist orientation and tilted relative to the reference orientation. FIG. 11A shows the distal end tilted relative to the reference orientation and not twisted relative to the reference twist orientation. FIG. 11B and FIG. 11C show relative twist and two different amounts of tilt relative to the reference orientation.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this disclosure are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. An endoscope shape and distal end orientation tracking system, comprising:
a first sensor unit configured to be disposed at a distal end of an endoscope and generate position and orientation tracking data for the distal end of the endoscope;
a plurality of second sensor units, each of the second sensor units being configured to be disposed a one of a corresponding plurality of locations along a length of the endoscope proximal to the distal end of the endoscope and generate position tracking data for the respective location; and
a control unit configured to:
(1) receive (a) the position and orientation tracking data generated by the first sensor unit for the distal end of the endoscope, and (b) the position tracking data for each of the respective locations by the respective second sensor units;
(2) determine shape of the endoscope and orientation of the distal end of the endoscope based on the data generated by the first and second sensor units; and
(3) generate output to a display unit that causes the display unit to display a representation of the shape of the endoscope and orientation of the distal end of the endoscope.

2. The system of claim 1, wherein:
the first sensor unit comprises an accelerometer, a magnetometer, and a gyroscope that generate the position and orientation tracking data for the distal end of the endoscope; and
each of the plurality of second sensor units comprises an accelerometer and a magnetometer that generate the position tracking data for the respective location.

3. The system of claim 1, wherein the control unit stores calibration data used to determine the shape of the endoscope and orientation of the distal end of the endoscope from the data generated by the first sensor unit and the second sensor units.

4. The system of claim 1, further comprising:
one or more wireless transmitters to wirelessly transmit: (1) the position and orientation tracking data generated by the first sensor unit for the distal end of the endoscope, and (2) the position tracking data for the plurality of locations generated by the second sensor units; and
wherein the control unit includes a wireless receiver to receive the data transmitted by the one or more wireless transmitters.

5. The system of claim 4, wherein each of the first sensor unit and the plurality of the second sensor units comprises one of the wireless transmitters.

6. The system of claim 1, comprising an insertion wire assembly comprising an insertion wire, the first sensor unit coupled with the insertion wire, and the second sensor units coupled with the insertion wire, the insertion wire assembly being configured for insertion into a working channel of the endoscope to position the first sensor unit adjacent to the distal end of the endoscope and each of the second sensor units at a respective one of the locations along the length of the endoscope, the insertion wire assembly being removable from the working channel when the distal end of the endoscope is disposed within a patient.

7. The system of claim 1, wherein each of the first sensor unit and each of the second sensor units is a disposable unit shaped for attachment to an external surface of the endoscope.

8. The system of claim 7, wherein each of the first sensor unit and each of the second sensor units comprises one of the wireless transmitters.

9. The system of claim 8, wherein each of the first sensor unit and each of the second sensor units comprises a battery.

10. The system of claim 1, wherein each of the first sensor unit and the plurality of the second sensor units are embedded within the endoscope during manufacturing of the endoscope.

11. The system of claim 1, wherein the displayed representation of the shape of the endoscope and orientation of the distal end of the endoscope indicates:
a longitudinal twist angle of the distal end of the endoscope relative to a reference twist angle; and
the amount of tilt of the distal end of the endoscope.

12. The system of claim 11, wherein the displayed representation of the orientation of the distal end of the endoscope displays the amount of tilt of the distal end of the endoscope via a representation that is rotated by an angle matching the longitudinal twist angle relative to a reference display angle.

13. The system of claim 11, wherein the displayed representation of the shape of the endoscope and orientation of the distal end of the endoscope includes a three-dimensional representation of the distal end of the endoscope as viewed from a viewpoint that varies to reflect changes in the orientation of the distal end of the endoscope.

14. A method for tracking shape and distal end orientation of an endoscope, the method including:
generating position and orientation tracking data for the distal end of an endoscope with a first sensor unit disposed at the distal end of the endoscope;
transmitting the position and orientation tracking data for the distal end of the endoscope from the first sensor unit to a control unit;
generating position tracking data for each of a plurality of locations along a length of the endoscope proximal to the distal end of the endoscope with a plurality of second sensors, each of the second sensors being disposed at a respective one of the locations along the length of the endoscope;
transmitting the position tracking data for the locations along the length of the endoscope from the second sensors to the control unit;
processing the position and orientation tracking data for the distal end of the endoscope and the position tracking data for the locations along the length of the endoscope with a control unit to determine shape of the endoscope and orientation of the distal end of the endoscope; and
generating output to a display unit that causes the display unit to display a representation of the shape of the endoscope and orientation of the distal end of the endoscope.

15. The method of claim 14, wherein generating position and orientation tracking data for the distal end of an endoscope comprises:
measuring accelerations of the first sensor unit via an accelerometer included in the first sensor unit; and
measuring orientation of the first sensor unit via a magnetometer included in the first sensor unit and/or a gyroscope included in the first sensor unit.

16. The method of claim 14, wherein generating position tracking data for the locations along the length of the endoscope comprises measuring accelerations of each of the second sensor units via an accelerometer included in the respective second sensor unit.

17. The method of claim 14, wherein:
transmitting the position and orientation tracking data for the distal end of the endoscope from the first sensor unit to a control unit comprises wireless transmitting the position and orientation tracking data from the first sensor unit and receiving the wirelessly transmitted position and orientation tracking data via a wireless receiver included in the control unit; and
transmitting the position tracking data for the locations along the length of the endoscope from the second sensors to the control unit comprises wireless transmitting the position tracking data from the second sensor units and receiving the wirelessly transmitted position tracking data via a wireless receiver included in the control unit.

18. The method of claim 14, comprising inserting an insertion wire assembly into a working channel of the endoscope, the insertion wire assembly including an insertion wire, the first sensor unit coupled with the insertion wire, and the second sensor units coupled with the insertion wire.

19. The method of claim 14, comprising attaching the first sensor unit and the second sensor units to an exterior surface of the endoscope.

20. The method of claim 14, comprising displaying a three-dimensional representation of the distal end of the endoscope as viewed from a viewpoint that varies to reflect changes in the orientation of the distal end of the endoscope.
